(19) United States
(12) Patent Application Publication
(10) Pub. No.: US 2022/0118044 A1
(43) Pub. Date: Apr. 21, 2022

(54) Injectable garlic formulation and a method of using the injectable garlic formulation

(71) Applicant: David Xiage Gan, Rockville, MD (US)

(72) Inventor: David Xiage Gan, Rockville, MD (US)

(21) Appl. No.: 17/072,732

(22) Filed: Oct. 16, 2020

(51) Int. Cl. A61K 36/8962; A61K 9/00; A61K 45/06; A61P 17/04

(52) U.S. Cl. CPC A61K 36/8962; A61P 17/04; A61K 45/06; A61K 9/0019

(57) ABSTRACT

An injectable garlic formulation that is readily used for administration in mammals to treat diseases. The garlic formulation contains pharmaceutically effective amount of water extract of raw garlic (WERG) or organic phase of raw garlic extract (OPRGE) constituted in pharmaceutically acceptable solvents. A method of treating a disease in a mammal by administering a pharmaceutically effective amount of the injectable garlic formulation thereof to the mammal. The garlic formulation is administered by direct injection or infusion prepared in pharmaceutically acceptable infusion buffer solution. The diseases to be treated include infectious diseases, inflammatory diseases, diabetes, cardiovascular diseases, and malignant tumors.

### CROSS REFERENCE TO RELATED APPLICATIONS (IF ANY)

(Related applications may be listed on an application data sheet, either instead of or together with being listed in the specification.)

No

### STATEMENT OF FEDERALLY SPONSORED RESEARCH OR DEVELOPMENT (IF ANY)

No Federally Sponsored Research or development.

### THE NAMES OF THE PARTIES TO A JOINT RESEARCH AGREEMENT IF THE CLAIMED INVENTION WAS MADE AS A RESULT OF ACTIVITIES WITHIN THE SCOPE OF A JOINT RESEARCH AGREEMENT

No joint research agreement.

### BACKGROUND OF THE INVENTION

### Field of Invention

The present invention is related to an injectable garlic formulation that is readily used for administration in mammals to treat diseases. The garlic formulation contains pharmaceutically effective amount of water extract of raw garlic (WERG) or organic phase of raw garlic extract (OPRGE) constituted in pharmaceutically acceptable solvents. The injectable garlic formulation has antimicrobial, anti-inflammatory, anti-diabetic, cardiovascular function-protecting, and anti-tumor activities.

The present invention is also related to a method of treating a disease in a mammal by administering a pharmaceutically effective amount of the injectable garlic formulation described above to the mammal. The garlic formulation is administered by direct injection or infusion prepared in pharmaceutically acceptable infusion buffer solution. The diseases to be treated include infectious disease, inflammatory diseases, diabetes, cardiovascular diseases, and malignant tumors.

### Background of Invention

The uses of garlic ((Allium sativum L.), consumed as raw garlic or garlic extract, to treat diseases, including but not limited to, heart conditions, headaches, bites, worms, wounds, ulcers, and tumors, can be dated back to 3000 BC (Freeman et al., 1995). The benefits of garlic are credited to antimicrobial (antibacterial, antifungal, and antiviral), cardiovascular function-protecting, anticancer, anti-hyperglycemic, insecticidal, anti-parasitic, and anti-inflammatory effects of organosulfur compounds presented in or derived from garlic (Hughes et al., 1991; Marchese et al. 2016; Bayan et al., 2014). Among those organosulfur compounds, the allicin (also known as S-(2-propenyl) 2-propene-1-sulfinothioate or diallyl thiosulfinate) is not presented in the intact garlic or garlic powder. The allicin is produced during crushing, mincing, or blending of garlic clover or wetting of garlic powder, which activates endogenous alliinase to convert endogenous alliin (a nonprotein amino acid S-allylcysteine S-oxide) predominantly to allicin (Lawson et al., 2005; Freeman et al., 1995). The other organosulfur compounds, such as bis(2-propenyl) 2-propenyl disulfide (also called diallyl disulfide), bis(2-propenyl) 2-propenyl trisulfide, (Z)- and (E)-ajoene (4,5,9-trithiadodeca-1,6,11-triene 9-oxide) are derived from allicin also during preparation of garlic extract.

Although the benefits of garlic have been widely examined, the in vivo evidence of garlic in treating diseases has not been consistent. Several reasons may contribute to this inconsistency. Supplemental garlic powder or extract is taken orally and the allicin released or produced while travelling down the digestive tract binds to the lumen, which prevents the allicin from entering the blood stream. Also, allicin is highly unstable and is converted to diallyl sulfide, diallyl disulfide, diallyl trisulfide, allyl methyl trisulfide, dithiins and/or ajoene during the preparation of garlic supplement or metabolic process in the body (Marchese et al. 2016). The method of preparing garlic powder or extract (aqueous or organic) further determines the final content of organosulfur compounds and/or antimicrobial effects (Hughes et al., 1991; Khan et al., 2000; Nikolic et al., 2004; Lawson et al., 2005). Administrations of garlic extract by injection have been also reported (Hunan Medical College, 1980; Davis et al., 1990; Li et al., 2018); however, the amount of garlic extract for administration cannot be accurately quantified because the amount is either estimated from the wet weight of the raw garlic or from proprietary oily extract. Thus, a standardized method to obtain garlic extract for injection is also described in the present invention here.

Currently, the most common types of cancer treatment are chemotherapy, radiotherapy, tumor surgery, in the case of prostate cancer and breast cancer—hormonal therapy, and newly developed immunotherapy. Innovations in cancer treatment aim to address a set of issues that healthcare providers and patients will typically face, including aggressive treatment accompanied by unwanted side effects, tumor recurrence after treatment, surgery, or both, and aggressive cancers that are resilient to widely utilized treatments.

To treat bacterial infections, a variety of antibiotics or antibacterial drugs is available. However, the over-prescription of antibiotics in the past half century gives rise to antibiotic-resistant strains of bacteria such as superbugs. Antibiotic-resistant microorganisms are increasing at an alarming rate. A list of infections, i.e., pneumonia, tuberculosis, and gonorrhea that are becoming harder and at times impossible to treat, is growing because antibiotics are less effective over the time. Antibiotic-resistant infections correlate with the level of antibiotic consumption. Non-judicial use of antibiotics is mostly responsible for making the microbes resistant. The antibiotic treatment repertoire for existing or emerging hard-to-treat multidrug-resistant bacterial infections is limited, resulting in high morbidity and mortality (Bin Zaman et al., 2017).

Type 2 diabetes mellitus (T2DM) is a global pandemic, chronic, progressive, incompletely understood metabolic condition chiefly characterized by hyperglycemia. Impaired insulin secretion, resistance to tissue actions of insulin, or a combination of both is thought to be the most common reason contributing to the pathophysiology of T2DM, a spectrum of disease originally arising from tissue insulin resistance and gradually progressing to a state characterized by complete loss of secretory activity of the beta cells of the pancreas (Chaudhury et al., 2017).

For viral infections in general or upper respiratory viral infections, effective drugs to hinder reproduction of viruses are much less available. Prescription of anti-viral drugs may reduce complications such as pneumonia, but often viruses are resistant to drugs.

### BRIEF SUMMARY OF THE INVENTION

The present invention relates to an injectable garlic formulation containing pharmaceutically effective amount of water extract of raw garlic (WERG) or organic phase of raw garlic extract (OPRGE) for administration in mammals. The injectable garlic formulation has antimicrobial, anti-inflammatory, anti-diabetic, cardiovascular function-protecting, and anti-tumor activities.

The present invention also relates to a method of treating a disease, including but not limited to infectious disease, inflammatory diseases, diabetes, cardiovascular diseases, and malignant tumors, in a mammal, the method comprising administering a pharmaceutically effective amount of the injectable garlic formulation containing pharmaceutically effective amount of water extract of raw garlic (WERG) or organic phase of raw garlic extract (OPRGE) to the mammal.

The pharmaceutically effective amount of water extract of raw garlic (WERG) or organic phase of raw garlic extract (OPRGE) is consistently prepared from fresh garlic (Allium sativum, all species) and validated by high performance liquid chromatography (HPLC), and is tolerated by mammal without detrimental adverse effects. The WERG is stored in a dehydrated form to enhance shelf life and is constituted in pharmaceutically acceptable solvent, such as water or buffer, before use. The OPRGE is dissolved in pharmaceutically acceptable organic solvent, such as dimethyl sulfoxide (DMSO) or Tween 80, and is aliquoted to target concentration for short-term or long-term storage in a refrigerator or freezer, respectively. Organosulfur compounds, including but not limited to allicin, ajoene, allyl methyl thio-sulfinate, methyl allyl thio-sulfinate are detected in the extract consistent with the report by Lawson et al., 2005, which is incorporated in its entirety here. Individual organosulfur compounds can be purified from garlic extractions or chemically synthesized. Two or more of these organosulfur compounds can be subsequently mixed to prepare the injectable garlic formulation.

The routes of injection include but not limited to intravenous (IV), intraperitoneal (IP), intramuscular (IM), and intra-tumor (IT) injection. For intravenous injection, infusion or intravenous drip is preferred to minimize adverse effects. The infusion or intravenous drip lasts 1 to 4 hours.

### DETAILED DESCRIPTION OF THE INVENTION

An injectable garlic formulation containing pharmaceutically effective amount of water extract of raw garlic (WERG) or organic phase of raw garlic extract (OPRGE) for administration in mammals. The WERG is first prepared by blending the cloves of fresh raw garlic in distilled water at a 1:9 weight/volume ratio, followed by filtration to remove large debris and centrifugation to obtain clear supernatant, consistent with the procedure of Li et al., 2018, which is incorporated in its entirety here. To increase the shelf-life and to standardize the content of extract, the clear supernatant is subsequently sterilized through a 0.22 μm filter unit, aliquoted, and finally lyophilized (freeze-drying at −80° C.) to obtain a dehydrated WERG for long-term storage. The OPRGE is prepared based on hydrophobicity as described by Li et al., 2018 and thus incorporated in its entirety here. An equal volume of chloroform/methanol (1:1) is added to the clear supernatant of WERG above. After thorough homogenization, an equal volume of 1-butanol/50 mM NaCl (4:5, v/v) mixture is added, followed by vortexing and/or sonicating, and centrifuging to obtain 3 phases: a colorless upper aqueous phase; a white solid interphase; and a light yellowish lower organic phase. To increase the shelf life and to standardize the content of extract, the organic phase is collected carefully, dried by filtering through sodium sulfate, followed by evaporation in a rotary evaporator to obtain a viscous fluid. The evaporated viscous fluid is dissolved in pharmaceutically acceptable organic solvent, sterilized through a 0.22 μm filter unit, aliquoted, and finally stored at −20° C. or −80° C. sealed with nitrogen gas for up to 12 months.

The injectable garlic formulations show antimicrobial, anti-inflammatory, anti-diabetic, cardiovascular function-protecting, and anti-tumor activities in mammals, including humans and animals, such as mice, rats, cats, and dogs, with no detectable or minimal adverse effects.

A method of treating a disease, including but not limited to infectious diseases, inflammatory diseases, diabetes, cardiovascular diseases, and malignant tumors, in a mammal, the method comprising administering a pharmaceutically effective amount of the injectable garlic formulation to the mammal. The injectable garlic formulation containing pharmaceutically effective amount of water extract of raw garlic (WERG) or organic phase of raw garlic extract (OPRGE), described in this invention.

The pharmaceutically effective amount of water extract of raw garlic (WERG) or organic phase of raw garlic extract (OPRGE) is prepared from fresh garlic (Allium sativum, all species) with proper conditions as described above to produce consistent contents of organosulfur compounds for long-term storage. The content of organosulfur compounds is validated by high performance liquid chromatography (HPLC). The pharmaceutically effective amount is also acceptable to the mammals without detrimental adverse effects. The WERG is stored in a dehydrated form to enhance shelf life and is constituted in pharmaceutically acceptable solvent, such as water or buffer, before use. The OPRGE is dissolved in pharmaceutically acceptable organic solvent, including but not limited to dimethyl sulfoxide (DMSO) or Tween 80, and is aliquoted to target concentration for short-term or long-term storage in a refrigerator or freezer, respectively. Organosulfur compounds, including but not limited to allicin, ajoene, allyl methyl thio-sulfinate, methyl allyl thio-sulfinate are detected in the extract. Individual organosulfur compounds can be purified from garlic extractions or chemically synthesized. Two or more of these organosulfur compounds can be subsequently mixed to prepare an injectable garlic formulation. HPLC analysis revealed more than 45% of organosulfur compounds in WERG or OPRGE are allicin and ajoene.

Thus, the “WERG” is defined here as dehydrated extract prepared by the method described in this invention and measured by HPLC to contain allicin and ajoene in a ratio of about 1:3 to 1:5. The “OPRGE” is defined here as evaporated extract prepared by the method described in this invention and measured by HPLC to contain allicin and ajoene in a ratio of about 1:3 to 1:5. In one embodiment, the combination of allicin and ajoene makes up about 10% to 20% of detectable compounds in WERG and about 40% to 60% of detectable compounds in OPRGE. The pharmaceutically effective amount is also defined as the amount that is effective to treat diseases without detrimental side or adverse effects. The effective amount of WERG ranges from 0.05 mg/kg to 10 mg/kg, from 0.1 mg/kg to 10 mg/kg, or from 0.5 mg/kg to 6 mg/kg daily. The effective amount of OPRGE ranges from 0.01 mg/kg to 1 mg/kg, 0.05 mg/kg to 1 mg/kg, or 0.1 mg/kg to 0.5 mg/kg daily. The injection can be administered daily for at least one day, for examples several days per week or per month continuously or intermittently.

The routes of injection include but not limited to intravenous (IV), intraperitoneal (IP), intramuscular (IM), and intra-tumor (IT) injection. For intravenous injection, infusion or intravenous drip is preferred to minimize adverse effects. The volume of each injection is less than 5 ml for direct injection and is 200 to 1000 ml for infusion or intravenous drip. The infusion or intravenous drip takes 1 to 4 hours.

The injectable garlic formulation can be administered to a patient while the patient is on other antimicrobial, anti-inflammatory, anti-diabetic, cardiovascular, and/or anti-cancer medications to provide synergistic effects on the treatment of diseases. The other medications include but not limited to omeprazole (e.g., for treating Helicobacter pylori); ß-lactams (e.g., for inhibiting Staphylococcus aureus, S. epidermidis, or P. aeruginosa); vancomycin (e.g., for suppressing vancomycin-resistant enterococci); polymyxin B or amphotericin B (e.g., for antifungal infection); prostacyclin, forskolin, indomethacin, or dypiridamole (e.g, for anti-platelet aggregation); and eicosapentaenoic acid (e.g., for anti-cancer treatment).

The terms of “cancers”, “malignant neoplasms”, and “malignant tumors” as used herein are interchangeable. The cancers or malignant tumors include but not limited to breast, blood (e.g., chronic myelocytic leukemia), bladder, gastric, oral cavity, colorectal, skin, uterus, cervical, esophagus, and lung cancers; and sarcoma.

The anti-hyperglycemic and anti-lipidemic effects of the present invention are also applied to treat diabetes (Type I or Type II), metabolic syndromes, and hyperlipidemia.

The antimicrobial activity of the present invention is used to inhibit i) Gram-negative and Gram-positive bacteria, including multidrug-resistant enterotoxigenic strains of Escherichia coli or resistant Staphylococcus aureus; ii) fungal infection, including Candida albicans; iii) intestinal protozoan parasites such as Entamoeba histolytica and Giardia lamblia; and iv) viruses, including cytomegalovirus, influenza A or B virus, herpes simplex virus type 1 and type 2, parainfluenza virus type 3, rhinoviruses, and rotaviruses. In one embodiment, the infectious disease is COVID-19 infection. In other embodiment, the inflammatory diseases include eczema, atopic dermatitis, and neurodermatitis.

For intravenous infusion, the hydrated WERG or aliquoted OPRGE in pharmaceutically acceptable organic solvent is dissolved in the following intravenous buffer solutions, including but not limited to.
0.9% Normal Saline (also known as NS, 0.9NaCl, or NSS)
Lactated Ringers (also known as LR, Ringers Lactate, or RL)
5% Dextrose in Water (also known as D5 or D5W)
0.45% Normal Saline (also known as Half Normal Saline, 0.45NaCl)

### EXAMPLES

### Example 1

For each 1 g of raw garlic, about 200 mg of dehydrated WERG and about 1.5 mg of evaporated OPRGE were obtained. The content of organosulfur compounds in WERG, determined by HPLC, includes allicin and ajoene in a ratio of about 1:4. The combination of allicin and ajoene makes up about 14% of detectable peaks in WERG. The content of organosulfur compounds in OPRGE, determined by HPLC, includes allicin and ajoene in a ratio of about 1:4. The combination of allicin and ajoene makes up about 48% of detectable peaks in OPRGE.

### Example 2

Case 1 study: A male adult (65 kg body weight) developed eczema on the left index finger. The rash and itchiness worsen in the next three days. On day five, WERG (400 mg in 500 ml normal saline) was administered by intravenous infusion. Adverse symptoms consistent with hypoglycemia start to develop at or after infusion of 350 ml. Upon completion of the 500 ml infusion and two regular bowls of rice porridge were given. The adverse symptoms disappeared within one hour. The rash from eczema was cleared next day.

Case 2 study: A male adult (65 kg body weight) had 8 lesions from neurodermatitis on the right forearm at the elbow area for over a year. Many topical medications from local pharmacy were applied but the lesions had never been totally cleared. Three-day regimen consisting of infusion of WERG (400 mg in 500 normal saline daily) for the first two days followed by infusion of OPRGE (15 mg first dissolved in 3 ml DMSO and then added to 500 ml normal saline) on day 3 was given. The infusion lasts 1.5 to 4 hours in each administration. Adverse symptoms consistent with hypoglycemia start to develop at or after infusion of 350 ml WERG. After taking two bowls of rice porridge or two cups of yogurt, the adverse symptoms disappeared within one hour. The infusion of OPRGE did not have the symptoms of hypoglycemia. One week after the three-day regimen, redness of the lesions start to fade away. The scars from the lesions disappeared after three months.

### Example 3

The toxicity or allergy studies of injectable OPRGE and WERG formulations in animals:

| | Route, | | |
|---|---|---|---|
| Animals | frequency | Dosage | Adverse effects |
| The | | | |
| Studies of | | | |
| OPRGE | | | |
| Beagle | Intravenous | (a) 6 mg/kg | Group (a): Tongue extension; |
| Dogs (1 | bolus, single | (b) 12 mg/kg | saliva secretion; and mild |
| in each | | | vomiting. |
| group) | | | Group (b): Tongue extension; |
| | | | saliva secretion; unable to |
| | | | stand; breathing deeply; and |
| | | | frequent vomiting. |
| Guinea | Intraperitoneal | (a) sensitized with 1.08 | No apparent allergic reactions |
| Pigs (4 | sensitization | mg/kg, then excited with | in group (a), (b), (c), or (d). |
| in each | (every other day, | 0.36 mg/kg (day 14) | |
| group) | 3x), then | (b) 1.08 mg/kg, then | |
| | intravenous bolus | excited with 0.36 mg/kg | |
| | excitation on day | (day 21) | |
| | 14 or 21 post- | (c) sensitized with 0.36 | |
| | sensitization | mg/kg, then excited with | |
| | | 0.12 mg/kg (day 14) | |
| | | (d) sensitized with 0.36 | |
| | | mg/kg, then excited with | |
| | | 0.12 mg/kg (day 21) | |
| Sprague- | Intravenous | (a) 11.4 mg/kg (day 0), | Group (a): Reduced activity; |
| Dawley | bolus, daily for 7 | then 7.98 mg/kg (days | vertical hairs; hematuria; and |
| Rats (8 | days | 1-6) | abnormal blood count. |
| in each | | (b) 5.7 mg/kg | Group (b): Reduced activity |
| group) | | | and vertical hairs. |
| Sprague- | Intravenous | (a) 0.4mg/kg | No apparent adverse effects |
| Dawley | bolus, daily for 7 | (b) 0.8mg/kg | |
| Rats (8 | days | | |
| in each | | | |
| group) | | | |
| The | | | |
| Studies of | | | |
| WERG | | | |
| Beagle | Intravenous | (a) 2000 mg/kg (dead on | Unable to land on its |
| Dogs (1 | bolus, single | day 0) | forelimbs; vomiting; and |
| in each | | (b) 1000 mg/kg | abnormal electrocardiogram |
| group) | | | and liver function in both |
| | | | groups (a) and (b) |
| Beagle | Intravenous drip, | (a) 630 mg/kg (days 0 to | Intermittent tremor; deepening |
| Dogs (2 | daily for 8 days | 4), then 315 mg/kg (days | of breathing; vomiting; |
| in each | | 5 to 7) | abnormal liver function; and |
| group) | | | hypokalemia |
| Guinea | Intraperitoneal | (a) sensitized with 180 | Allergic reactions including |
| Pigs (4 | sensitization | mg/kg, then excited with | restlessness, nasal scratching, |
| in each | (every other day, | 60 mg/kg (4 dead on day | unstable gait, dyspnea, and |
| group) | 3x), then | 0) | cramps in groups (a), (b), (c), |
| | intravenous bolus | (b) sensitized with 60 | and (d) |
| | excitation on day | mg/kg, then excited with | |
| | 14 post- | 20 mg/kg (4 dead on day | |
| | sensitization | 0) | |
| | | (c) sensitized with 180 | |
| | | mg/kg, then excited with | |
| | | 20 mg/kg (4 dead on day | |
| | | 0) | |
| | | (d) sensitized with 60 | |
| | | mg/kg, then excited with | |
| | | 6.7 mg/kg (2 dead on day | |
| | | 0) | |
| Sprague- | Intravenous | (a) 1900 mg/kg (3 dead | Accelerated breathing, |
| Dawley | bolus, daily for 7 | on day 0), then 1330 | drowsiness, vertical hairs, and |
| Rats (8 | days | mg/kg (days 1-6, 2 | fluctuation of blood glucose |
| in each | | dead on day 2) | (hypoglycemia or |
| group) | | (b) 950 mg/kg (1 dead | hyperglycemia) in both groups |
| | | on day 3) | (a) and (b) |
| Sprague- | Intravenous | (a) 34 mg/kg | No apparent adverse effects |
| Dawley | bolus, daily for 7 | (b) 67 mg/kg | |
| Rats (8 | days | | |
| in each | | (c) 134 mg/kg | |
| group) | | | |

1. An injectable garlic formulation consisting of pharmaceutically effective amount of water extract of raw garlic (WERG) or organic phase of raw garlic extract (OPRGE) and pharmaceutically acceptable solvent, and optionally a pharmaceutically acceptable buffer for administration in mammals, wherein the effective amount is 0.1 to 10 mg/kg daily for WERG and is 0.05 to 1 mg/kg daily for OPRGE.

2. A method of treating a disease in a mammal, the method comprising administering to the mammal in need thereof a pharmaceutically effective amount of an injectable garlic formulation consisting of pharmaceutically effective amount of water extract of raw garlic (WERG) or organic phase of raw garlic extract (OPRGE) and pharmaceutically acceptable solvent, and optionally a pharmaceutically acceptable buffer for administration in mammals, wherein the effective amount is 0.1 to 10 mg/kg daily for WERG and is 0.05 to 1 mg/kg daily for OPRGE.

3. The method according to claim 2, wherein the disease is selected from the group consisting of infectious diseases, inflammatory diseases, diabetes, cardiovascular diseases, skin diseases and cancers.

4. The method according to claim 3, wherein the method further comprising administering concomitantly or intermittently additional agents for treating infectious diseases, inflammatory diseases, diabetes, cardiovascular diseases, skin diseases, or cancers.

5. The method according to claim 2, wherein the WERG or OPRGE comprises an organosulfur compound selected from the group consisting of allicin, ajoene, allyl methyl thio-sulfinate, methyl allyl thio-sulfinate, and combinations thereof.

6. The method according to claim 2, wherein the garlic is all species of garlic Allium sativum.

7. The method according to claim 2, wherein the effective amount of OPRGE is 3 mg to 20 mg daily and administered for one week to treat infectious diseases or skin diseases.

8. The method according to claim 7, wherein the infectious disease is COVID-19 (Sars-Cov-2 virus), bacterial, fungal, or parasite infection.

9. The method according to claim 2, wherein the effective amount of OPRGE is 10 mg to 50 mg daily and administered for 3 weeks to treat cancers.

10. The method according to claim 2, wherein the effective amount of OPRGE is 5 mg to 20 mg daily and administered for 4 weeks to treat type 2 diabetes.

11. A method of treating a disease in a mammal, the method comprising administering to the mammal in need thereof a pharmaceutically effective amount of an injectable garlic formulation consisting of pharmaceutically effective amount of two or more organosulfur compounds identified in water extract of raw garlic (WERG) or organic phase of raw garlic extract (OPRGE) and pharmaceutically acceptable solvent, and optionally a pharmaceutically acceptable buffer for administration in mammals, wherein the two or more organosulfur compounds are purified or chemically synthesized individually and the effective amount is 0.01 to 1 mg/kg daily.

12. The method according to claim 11, wherein the disease is selected from the group consisting of infectious diseases, inflammatory diseases, diabetes, cardiovascular diseases, skin diseases, and cancers.

13. The method according to claim 12, wherein the method further comprising administering concomitantly or intermittently additional agents for treating infectious diseases, inflammatory diseases, diabetes, cardiovascular diseases, skin diseases, or cancers.

14. The method according to claim 12, wherein the infectious disease is COVID-19 (Sars-Cov-2 virus), bacterial, fungal, or parasite infection.
